(19) United States
(12) Patent Application Publication
(10) Pub. No.: US 2006/0251685 A1
(43) Pub. Date: Nov. 9, 2006

(54) Stable ophthalmic oil-in-water emulsions with Omega-3 fatty acids for alleviating dry eye

(76) Inventors: Zhi-Jian Yu, Irvine, CA (US); Lauren Crawford, Mission Viejo, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/418,486

(22) Filed: May 3, 2006

Related U.S. Application Data
(63) Continuation-in-part of application No. 11/098,827, filed on Apr. 4, 2005.
Application No. 11/098,827 is a continuation-in-part of application No. 10/802,153, filed on Mar. 17, 2004.
Application No. 10/802,153 is a continuation-in-part of application No. 10/392,375, filed on Mar. 18, 2003.

(51) Int. Cl. A61K 36/53; A61K 9/00

(52) U.S. Cl. 424/400; 424/745

(57) ABSTRACT

An ophthalmic composition includes oil globules dispersed in an aqueous phase. The globules include a surfactant component, a polar oil component that includes an Omega-3 fatty acid and a viscosity modifying agent. The surfactant to oil ratio produces an average size of globules of about 0.1 microns or less. The viscosity is at least as viscous as 0.25% 800K sodium hyaluronate. The composition can be used for treatment of dry eye. The compositions are stable and can have anti-microbial activity sufficient for use as contact lens disinfecting solutions.

### RELATED APPLICATIONS

This application is a continuation-in-part of U.S. application Ser. No. 11/098,827, filed Apr. 4, 2005 which is a continuation-in-part U.S. application Ser. No. 10/802,153, filed Mar. 17, 2004 which is a continuation-in-part of U.S. application Ser. No. 10/392,375, filed Mar. 18, 2003. All three applications are incorporated herein by reference.

### BACKGROUND OF THE INVENTION

1. Field of the Invention

Embodiments of the invention relate to ophthalmic compositions containing Omega-3 Fatty Acids for the treatment and/or relief of dry eye.

2. Description of the Related Art

Dry eye syndrome is a prevalent condition for which there is no cure, although symptoms may be relieved with proper diagnosis and treatment. The condition affects more than 3.2 million American women middle-aged and older alone (Schaumberg D A, Sullivan D A, Buring J E, Dana M R. Prevalence of dry eye syndrome among US women. Am J Ophthalmol 2003 August;136(2):318-26). Contact lens wearers, computer users, patients who live and/or work in dry environments, and patients with autoimmune disease are all particularly susceptible to developing dry eye.

Omega-3 Fatty Acids have been shown to effectively treat symptoms of dry eye when taken orally. There is a need to create a solution that contains Omega-3 fatty acids in an emulsion. Emulsions have a milky appearance. If emulsion droplet sizes are very small, less than about 0.1 micron, the emulsion is clear and is called a microemulsion. Omega-3 fatty acids can be incorporated into a contact lens solution either as an emulsion or as a microemulsion. It is desirable to incorporate a preservative with the emulsion or mircroemulsion to prevent bacterial growth and deterioration of the solution. It is known that Oxidative preservatives and non-polymeric quaternary amines are not compatible with Omega-3 fatty acids.

Viscosity agents such as carboxymethylcellulose (“CMC”) or Sodium Hyaluronate are added to contact lens solutions to make them more comfortable to wear. It is known that when Omega-3 fatty acids are added to a solution that has high viscosity and high polymer concentrations, the solutions are only stable at relatively low concentrations of CMC or Sodium Hyaluronate. There is a need to create a stable solution that contains a viscosity agent and Omega-3 fatty acids to relieve dry eye.

### SUMMARY OF THE INVENTION

Embodiments of the invention are directed to a stable composition containing Omega-3 fatty acids and a viscosity agent to be used as a contact lens solution to treat dry eye. Mildly stable compositions according to embodiments of the invention contain oil globules having an average size of about 0.18 micron. More preferred embodiments include stable compositions that contain oil globules having an average size of less than 0.1 micron dispersed in an aqueous phase. Some embodiments include stable compositions that contain oil globules having an average size of less than 0.08 micron dispersed in an aqueous phase. Some embodiments include stable compositions that contain oil globules having an average size of less than 0.05 micron dispersed in an aqueous phase. These globules may include a surfactant component and a polar oil component, such as an Omega-3 fatty acid.

Preferred embodiments of the invention are also directed to combining a polymeric quaternary amine preservative and an Omega-3 oil emulsion.

In preferred embodiments, the stable composition includes a preservative that is a polymeric quartenary amine such as poly[dimethylimino-w-butene-1,4-diyl]chloride, alpha-[4-tris(2-hydroxyethyl)ammonium]-dichloride (Polyquaternium 1®), poly(oxyethyl(dimethyliminio)ethylene dmethyliminio)ethylene dichloride (WSCP®), polyhexamethylene biguanide (PHMB), polyaminopropyl biguanide (PAPB).

In preferred embodiments of the invention, the polymeric quartenary amine is polyhexamethylene biguanide (PHMB).

In preferred embodiments, the oil component of the composition includes flaxseed oil, Perilla seed oil or another natural or synthetic oil that is a source of Omega-3 fatty acids.

In preferred embodiments of the invention, the stable composition is a self-emulsifying solution.

In preferred embodiments, the surfactant component and the oil component are selected to self-emulsify when mixed without mechanical homogenization. In preferred embodiments, the surfactant component of the self-emulsifying composition includes one or two surfactants.

In preferred embodiments, the surfactant component has a hydrophobic portion which includes a first part oriented proximal to the aqueous phase that is larger than a second part of the hydrophobic portion of the surfactant component oriented towards the interior of the oil globule. More preferably, the surfactant component includes one surfactant with the first part of the hydrophobic portion of the surfactant that contains more atoms than the second part of the hydrophobic portion of the surfactant. In some preferred embodiments, the surfactant component includes two surfactants, a first of said surfactants including a first hydrophobic portion and a second of said surfactants including a second hydrophobic portion, said first hydrophobic portion having a longer chain length than the second hydrophobic portion.

In some embodiments, the self-emulsifying composition also includes an additional surfactant that does not interfere with self-emulsification.

In preferred embodiments, self-emulsifying composition includes a surfactant component which is (a) a compound having at least one ether formed from at least about 1 to 100 ethylene oxide units and at least one fatty alcohol chain having from at least about 12 to 22 carbon atoms; (b) a compound having at least one ester formed from at least about 1 to 100 ethylene oxide units and at least one fatty acid chain having from at least about 12 to 22 carbon atoms; (c) a compound having at least one ether, ester or amide formed from at least about 1 to 100 ethylene oxide units and at least one vitamin or vitamin derivative; and (d) combinations thereof which have no more than two surfactants. In a preferred embodiment, the surfactant component is Lumulse GRH-40 or TPGS.

In preferred embodiments the surfactant component is Lumulse GRH-40.

In embodiments of the composition the oil globules have an average size of about 1.0 to 0.18 micron or less.

In embodiments of the composition the oil globules have an average size of about 0.5 to 0.18 micron or less.

In preferred embodiments of the composition the oil globules have an average size of less than about 0.1 micron.

In some embodiments of the composition the oil globules have an average size of less than about 0.08 micron.

In some embodiments of the composition the oil globules have an average size of less than about 0.05 micron.

In preferred embodiments, the self-emulsifying composition may be used as a multipurpose solution for contact lenses.

Embodiments of the invention are directed to methods of treating an eye which includes the steps of administering any of the self-emulsifying compositions described above to an individual in need thereof. Preferably, the treatment is for dry eye. Preferably, the individual is a mammal.

Embodiments of the invention are directed to methods of preparing a composition containing Omega-3 fatty acids which may include the steps of preparing an oil phase which includes a polar oil that is a source of Omega-3 fatty acids, such as flaxseed or Perilla seed oil, or other natural or synthetic oil that is a source of Omega-3 fatty acids and a surfactant component, wherein the polar oil and the surfactant component in the oil phase are in the liquid state; preparing an aqueous phase at a temperature that permits self-emulsification; wherein the aqueous phase comprises a water soluble polymer; and mixing the oil phase and the aqueous phase to form an emulsion, without mechanical homogenization. The method may also include forming a milky paste or a clear viscous gel between the oil phase and a part of the aqueous phase and mixing the paste or gel with the rest of the aqueous phase to form a clear emulsion.

Further aspects, features and advantages of this invention will become apparent from the detailed description of the preferred embodiments which follow.

### DESCRIPTION OF THE DRAWING

These and other features of this invention will now be described with reference to the drawings of preferred embodiments which are intended to illustrate and not to limit the invention.

FIGS. 1A and 1B show a flow chart for the preparation of the ophthalmic self-emulsifying compositions described.

### DETAILED DESCRIPTION OF THE PREFERRED EMBODIMENT

Embodiments of the invention are directed to ophthalmic oil-in-water emulsions which contain Omega-3 fatty acids. The integration of emulsions containing Omega-3 fatty acids into contact lens care compositions, such as multi-purpose, re-wetting and other contact lens care compositions adds the additional utility or benefit of prevention and/or treatment of dry eye and provides lubrication to the lens and/or eye through mechanisms only emulsions can provide. Additional utilities or benefits provided by integrated emulsions in contact lens care compositions may include, without limitation, enhanced contact lens cleaning, prevention of contact lens water loss, inhibition of protein deposition on contact lenses and the like.

There are two problems with incorporation of Omega-3 fatty acids into ophthalmic oil-in-water solutions. The first problem is that when emulsion droplet sizes are too large, the emulsion is only stable at low viscosity and at low concentrations of water-soluble polymers. The second problem is maintaining sterility of oil-in-water ophthalmic solutions which contain Omega-3 fatty acids.

When oxidative preservatives and non-polymeric quaternary amines, such as CPC, Alexidine and stabilized ClO₂, are incorporated into emulsions that contain Omega-3 fatty acids, the oxidative preservatives and non-polymeric quaternary amines lose their antimicrobial activity due to interaction with the Omega-3 fatty acids. Thus, it may be difficult to maintain antimicrobial activity in the presence of oxidative preservatives and non-polymeric quaternary amines.

A need exists for stable ophthalmic emulsions containing Omega-3 fatty acids. Additionally, it is desirable for the compositions to be stable at high viscosity and at high concentrations water-soluble polymers. It is further desirable for such stable compositions to have antimicrobial activity so that the compositions can be maintained free of microbial contamination.

Embodiments of the present invention provide oil-in-water emulsions containing Omega-3 fatty acids with mean emulsion droplet sizes of about 1.0 to 0.18 micron. These emulsions appear milky even when they are stable because the emulsion droplet sizes are big enough that the droplets can be seen with the naked eye. These emulsions are thermodynamically unstable.

Preferred embodiments of the present invention provide oil-in-water emulsions containing Omega-3 fatty acids with mean emulsion droplet sizes of less than about 0.1 micron. These embodiments represent an example of a microemulsion. These ophthalmic compositions appear clear because the droplet sizes are so small that the emulsion droplets cannot be seen with the naked eye. These microemulsions are thermodynamically stable.

Emulsions containing Omega-3 fatty acids have a low surfactant to oil ratio for high comfort and employ fewer surfactants to achieve emulsification. In some embodiments of the invention polymeric quaternary amines are added to the solution as a preservative. Ophthalmic compositions according to the invention are stable and free of microbial growth for at least 6 months. These compositions can be designed to employ molecular self-assembly methods to generate macromolecular oil droplet structures at the nanometer scale, and thus represent an example of nanotechnology.

Definitions

The term “emulsion” is used in its customary sense to mean a stable and homogenous mixture of two liquids which do not normally mix such as oil and water.

The term “microemulsion” is used to mean a stable and homogenous mixture of two liquids which do not normally mix, such as oil and water that appear clear and that have mean emulsion droplet sizes of less than about 0.1 micron.

An “emulsifier” is a substance which aids the formation of an emulsion such as a surfactant. The terms “emulsifier” and “surfactant” are used interchangeably herein. In the context of the present invention, surfactant component means one or more surfactants that are present in the self-emulsifying composition and contribute to the self-emulsification.

The term “stable” is used in its customary sense and means the absence of creaming, flocculation, and phase separation.

The term “demulcent” is used in the usual sense and refers to an agent that relieves irritation of inflamed or abraded lens and/or eye surfaces.

The term “polar oil” means that the oil contains heteroatoms such as oxygen, nitrogen and sulfur in the hydrophobic part of the molecule.

A “multi-purpose composition,” as used herein, is useful for performing at least two functions, such as cleaning, rinsing, disinfecting, rewetting, lubricating, conditioning, soaking, storing and otherwise treating a contact lens, while the contact lens is out of the eye. Such multi-purpose compositions preferably are also useful for re-wetting and cleaning contact lenses while the lenses are in the eye. Products useful for re-wetting and cleaning contact lenses while the lenses are in the eye are often termed re-wetters or “in-the-eye” cleaners.

The term “cleaning” as used herein includes the loosening and/or removal of deposits and other contaminants from a contact lens with or without digital manipulation and with or without an accessory device that agitates the composition.

The term “re-wetting” as used herein refers to the addition of liquid over at least a part, for example, at least a substantial part, of at least the anterior surface of a contact lens.

The term “paste” as used herein refers to a semisolid preparation which does not flow.

The term “clear viscous gel” as used herein refers to a semisolid preparation that is clear and does not flow.

Therapeutic ophthalmic compositions for the treatment and/or relief of dry eye are disclosed. The ophthalmic compositions include oil-in-water emulsions, preferably self-emulsifying oil-in-water emulsions, along with Omega-3 fatty acids. Preferred embodiments of the invention include oil-in-water emulsions or microemulsions that contain Omega-3 fatty acids and a biocide to control microbial growth. Methods of preparing or making such compositions and methods of using such compositions are also disclosed. The present emulsion-containing compositions are relatively easily prepared and are storage-stable, for example, having a shelf life at about room temperature of at least about 6 months or more. In addition, the present compositions are advantageously easily sterilized, for example, using sterilizing filtration techniques, and eliminate, or at least substantially reduce, the opportunity or risk for microbial growth if the compositions become contaminated by inclusion of at least one anti-microbial agent.

Preferred embodiments are directed to compositions comprising oil-in-water emulsions for the treatment of dry eye. For this use, one would administer a composition as needed as determined by one skilled in the art. For example, ophthalmic demulcents such as carboxymethylcellulose, other cellulose polymers, dextran 70, gelatin, glycerine, polyethylene glycols (e.g., PEG 300 and PEG 400), polysorbate 80, propylene glycol, polyvinyl alcohol, povidone and the like and mixtures thereof, carbomers (e.g. carbopol RTM), polyvinyl alcohol, polyvinyl pyrrolidone, alginates, carrageenans, and guar, karaya, agarose, locust bean, tragacanth and xanthan gums may be used in the present ophthalmic compositions, for example, compositions useful for treating dry eye.

Embodiments are directed to emulsions and microemulsions that contain Omega-3 fatty acids from flaxseed oil or Perilla seed oil.

Flaxseed oil is derived from Linum usitatissimum and has a very high level of alpha linolenic acid. Flaxseed oil has a maximum acid value of 2 mg KOH/g, a maximum peroxide value of 10 mEq/Kg, a minimum saponification value of 184 mg KOH/g and a maximum saponification value of 194 mg KOH/g, the specific gravity is a minimum of 0.927 g/mL at 20° C. and the color is 15 gardner. The minimum and maximum percentages for the fatty acid composition for flaxseed oil is indicated below.

Area %

| Fatty Acid Composition: | MIN | MAX |
|---|---|---|
| C 16:0 Palmitic Acid | 3 | 8 |
| C 18:0 Stearic Acid | 2 | 8 |
| C 18:1 Oleic Acid | 11 | 24 |
| C 18:2 Linoliec Acid | 11 | 24 |
| C 18:3 Gamma Linolenic Acid | 0 | 1 |
| C 18:3 Alpha Linolenic Acid | 45 | 65 |
| C 20:0 Icosanoic Acid | 0 | 1 |

Perilla seed oil has a maximum acid value of 5.0 mg KOH/g and a maximum Peroxide value of 5.0 mEq/Kg. The fatty acid composition for Perilla seed oil is indicated below.

Area %

| Fatty Acid Composition: | MIN | MAX |
|---|---|---|
| C 16:0 Palmitic Acid | | 10 |
| C 18:0 Stearic | | 5.0 |
| C 18:1 Oleic | 17 | |
| C 18:2 Linoleic | 13 | |
| C 18:3 Linolenic | 60 | |

The present compositions preferably include self-emulsifying emulsions. That is, the present oil-in-water emulsions preferably can be formed with reduced amounts of dispersion mixing at shear speed, more preferably with substantially no dispersion mixing at shear speed as further described in U.S. application Ser. Nos. 11/098,827, filed Apr. 4, 2005; Ser. No. 10/802,153, filed Mar. 17, 2004; and Ser. No. 10/392,375, filed Mar. 18, 2003.

Topical ophthalmic application forms of the present compositions include, without limitation, eye drops for dry eye treatment and for other treatments, and can also include forms for the delivery of drugs or therapeutic components into the eye and forms for caring for contact lenses. The present compositions are very useful for treating dry eye and similar conditions, and other eye conditions. In addition, the present compositions can be useful as carriers or vehicles for drug delivery, for example, a carrier or vehicle for delivery of therapeutic components into or through the eyes.

Contact lens care applications of the present compositions include, without limitation, compositions useful for cleaning, rinsing, disinfecting, storing, soaking, lubricating, re-wetting and otherwise treating contact lenses, including compositions which are effective in performing more than one of such functions, i.e., so called multi-purpose contact lens care compositions, other contact lens care-related compositions and the like. Contact lens care compositions including the present emulsions also include compositions which are administered to the eyes of contact lens wearers, for example, before, during and/or after the wearing of contact lenses.

Embodiments of the invention provide for therapeutic ophthalmic compositions which include oil-in-water emulsions, preferably self-emulsifying oil-in-water emulsions. These oil-in-water emulsions comprise an Omega-3 fatty acid component, for example, and without limitation, Perilla seed oil or flaxseed oil; and an aqueous component which includes two emulsifiers or surfactants or less. The use of only one or two emulsifiers results in a low weight ratio of emulsifying component to oil component and fewer chemical toxicity concerns, resulting in comfort and safety advantages over emulsions employing more than two emulsifiers.

The Omega-3 oily component and the surfactant component or surfactants are advantageously chemically structurally compatible to facilitate self-emulsification of the emulsion. In the context of the present invention, surfactant component means one or two surfactants that are present in the self-emulsifying composition and contribute to the self-emulsification. The one or two surfactants must have an affinity for the selected oil or oils based upon non-covalent bonding interactions between the hydrophobic structures of the surfactant and the oil(s) such that self emulsification can be achieved as further described in U.S. application Ser. Nos. 11/098,827, filed Apr. 4, 2005; Ser. No. 10/802,153, filed Mar. 17, 2004; and Ser. No. 10/392,375, filed Mar. 18, 2003.

In preferred embodiments, the one or two surfactants must be able to form a chemical structure which is wedge or pie section-shaped, with the larger end of the wedge structure closer to the hydrophilic parts of the surfactant structures as further described in U.S. application Ser. Nos. 11/098,827, filed Apr. 4, 2005; Ser. No. 10/802,153, filed Mar. 17, 2004; and Ser. No. 10/392,375, filed Mar. 18, 2003.

The surfactants useful to form the surfactant component in the present invention advantageously are water-soluble when used alone or as a mixture. These surfactants are preferably non-ionic. The amount of surfactant component present varies over a wide range depending on a number of factors, for example, the other components in the composition and the desired droplet emulsion sizes. The more surfactant is added, the smaller the droplet size. Often the total amount of surfactant component is in the range of about 0.01 to about 10.0 w/w %. It is noted that additional surfactant(s) may be present in the self-emulsifying composition (in addition to the surfactant component) and still fall within the scope of the present invention if the additional surfactant(s) are present at a concentration such that they do not interfere with the self-emulsification.

The ratio, for example, weight ratio, of the surfactant component to the oily component in the present oil-in-water emulsions is selected to provide acceptable emulsion stability and performance, preferably to provide a self-emulsifying oil-in-water emulsion, and preferably to create mean emulsion droplet sizes that are less than about 0.1 micron. Of course, the ratio of surfactant component to oily component varies depending on the specific surfactants and oil or oils employed, on the specific stability and performance properties desired for the final oil-in-water emulsion, on the specific application or use of the final oil-in-water emulsion and the like factors. For example, for an emulsion that contains mean droplet sizes of less than about 0.1 micron, the weight ratio of the surfactant component to the oily component may range from about 0.5 to 10.0, preferably from 1.0 to 5.0, more preferably from 2.0 to 4.0.

Such surfactants function as described herein, provide effective and useful ophthalmic compositions and do not have any substantial or significant detrimental effect on the contact lens being treated by the present compositions, on the wearers of such contact lenses or on the humans or animals to whom such compositions are administered.

One or more oils or oily substances are used to form the present compositions as illustrated in U.S. application Ser. Nos. 11/098,827, filed Apr. 4, 2005; Ser. No. 10/802,153, filed Mar. 17, 2004; and Ser. No. 10/392,375, filed Mar. 18, 2003. In preferred embodiments, oils that contain Omega-3 fatty acids are used. Flaxseed oil, Perilla oil and the other natural or synthetic oils are examples of sources of Omega-3 fatty acids.

Omega-3 fatty acids which are natural, safe, have prior ophthalmic or pharmaceutical use, have little color, do not easily discolor upon aging, easily form spread films and lubricate surfaces without tackiness are preferred. The compositions are comfortable and non-toxic to the eye.

The integration of oil-in-water emulsions with water soluble polymer demulcents into eye drops for dry eye treatment, contact lens rewetting and multipurpose solutions adds the additional utility of prevention of dry eye and contact lens water loss by providing an oil layer at the air-tear interface or additionally at the contact lens-tear interface when a contact lens is present. This oil layer acts to prevent dry eye or contact lens water loss by retarding water evaporation and thus loss. The oil layer on the surface of a contact lens can also provide a long-lasting lubrication layer, especially for rigid gas permeable contact lenses. The oil layer on the surface of a contact lens can also inhibit contact lens protein deposition.

The self-emulsifying, oil-in-water emulsions for the therapeutic compositions of the present invention are of two general types. The first type is a one surfactant system as illustrated in U.S. application Ser. Nos. 11/098,827, filed Apr. 4, 2005; Ser. No. 10/802,153, filed Mar. 17, 2004; and Ser. No. 10/392,375, filed Mar. 18, 2003. The second type is a two surfactant system, also illustrated in U.S. application Ser. Nos. 11/098,827, filed Apr. 4, 2005; Ser. No. 10/802,153, filed Mar. 17, 2004; and Ser. No. 10/392,375, filed Mar. 18, 2003.

As a practical matter, a surfactant is a good candidate for the self-emulsifying oil-in-water emulsions described herein if the surfactant is able to form droplets of a size of about 1.0 to 0.18 micron, preferably from 0.5 to 0.1 micron, more preferably less than about 0.1 micron.

Examples of one component surfactant systems include flaxseed oil or Perilla oil. A preferred example of a single surfactant and oil pair is the surfactant Lumulse GRH-40 and flaxseed oil. Another preferred example of a single surfactant and oil pair is Lumulse GRH-40 and Perilla oil.

Lumulse GRH-40 is a 40 mole ethoxylate of hydrogenated Castor oil as further explained in U.S. application Ser. Nos. 11/098,827, filed Apr. 4, 2005; Ser. No. 10/802,153, filed Mar. 17, 2004; and Ser. No. 10/392,375, filed Mar. 18, 2003.

The optimal amount of Lumulse GRH-40 to create an emulsion that contains mean droplet sizes of about 0.18 micron is about 1.5% w/w Lumulse GRH-40 and about 1.0% w/w flaxseed oil. Higher or lower amounts in conjunction with Omega-3 fatty acids can be used, however, depending upon the desired properties of the final emulsion. In general, the weight ratio of Lumulse GRH-40 to Omega-3 fatty acids is in the range of 0.5 to 10.0, preferably about 1.5.

The optimal amount of Lumulse GRH-40 to use to create a emulsion with a mean droplet size of less than 0.1 micron, in conjunction with flaxseed oil, is about 3.0% w/w Lumulse GRH-40 and about 1.0% w/w flaxseed oil. Higher or lower amounts in conjunction with Omega-3 fatty acids can be used, however, depending upon the desired properties of the final emulsion. In general, the weight ratio of Lumulse GRH-40 to Omega-3 fatty acids is in the range of 0.5 to 10.0, preferably about 3.0.

Lumulse GRH-40 can be combined with other surfactants such as Polysorbate-80 (Tween-80, polyoxyethylene (20) sorbitan mono-oleate) to create self-emulsifying emulsions comprised of two surfactants as further described U.S. application Ser. Nos. 11/098,827, filed Apr. 4, 2005; Ser. No. 10/802,153, filed Mar. 17, 2004; and Ser. No. 10/392,375, filed Mar. 18, 2003.

Embodiments of the invention are directed to a stable composition containing Omega-3 fatty acids to be used as a contact lens solution to treat dry eye. Mildly stable compositions according to embodiments of the invention contain oil globules having an average size of about 1.0 to 0.18 micron. Other embodiments contain oil droplets that contain oil droplets between about 0.5 to 0.18 micron. Preferred embodiments include stable compositions that contain oil globules having an average size of less than 0.1 micron dispersed in an aqueous phase. Some embodiments include stable compositions that contain oil globules having an average size of about 0.1 to 0.05 micron dispersed in an aqueous phase. These globules may include a surfactant component and a polar oil component.

Preferred embodiments of the invention contain at a minimum Omega-3 fatty acids and one surfactant and have an osmolality of 150 to 450 mOsm/kg, more preferably between about 250 to about 330 mOsm/kg, more preferably between about 270 to about 310 mOsm/kg and have a pH of 6.5 to 8.5, more preferably from about 7.3 to 7.7.

Two surfactants may also be selected to match a particular oil or oils with respect to the ability of the surfactants to form a self-emulsifying oil-in-water emulsion for the dry eye treatments according to the invention. Both surfactants must each meet two chemical structural requirements to achieve self emulsification: (1) each surfactant must have an affinity for the selected oil or oils based upon non-covalent bonding interactions between the hydrophobic structures of the surfactant and the oil(s) such that self emulsification can be achieved when requirement (2) is simultaneously met; and (2) the surfactant pair must be able to form a chemical structure which is wedge or pie section-shaped, with the larger end of the wedge structure closer to the hydrophilic parts of the surfactant structures as illustrated in U.S. application Ser. Nos. 11/098,827, filed Apr. 4, 2005; Ser. No. 10/802,153, filed Mar. 17, 2004; and Ser. No. 10/392,375, filed Mar. 18, 2003.

Additional surfactant(s) may be added which may or may not participate in emulsion formation.

Another example of a one component system utilizes a surfactant such as tocopherol polyethyleneglycol-succinate (TPGS, available from Eastman Chemical Company, Kingsport, Tenn.). TPGS can form a wedge with tocopherol in the narrow section, PEG in the outer section and succinate forming a covalent attachment between them.

More generic descriptions of the types of surfactants which can be used in the present invention include surfactants selected from: (a) at least one ether formed from 1 to 100 ethylene oxide units and at least one fatty alcohol chain having from 12 to 22 carbon atoms; (b) at least one ester formed from 1 to 100 ethylene oxide units and at least one fatty acid chain having from 12 to 22 carbon atoms; (c) at least one ether, ester or amide formed from 1 to 100 ethylene oxide units and at least one vitamin or vitamin derivative, and (d) mixtures of the above consisting of no more than two surfactants.

The preparation of the oil-in-water emulsions that contain Omega-3 fatty acids of the present invention is generally as follows. Non-emulsifying agents which are water soluble components are dissolved in the aqueous (water) phase and oil-soluble components including the emulsifying agents are dissolved in the oil phase. The two phases (oil and water) are separately heated to an appropriate temperature. This temperature is the same in both cases, generally a few degrees to 5 to 10 degrees above the melting point of the highest melting ingredients in the case of a solid or semi-solid oil or emulsifying agent in the oil phase. Where the oil phase is liquid at room temperature, a suitable temperature is determined by routine experimentation with the melting point of the highest melting ingredients in the aqueous phase. In cases where all components of either the oil or water phase are soluble in their respective phase at room temperature, no heating may be necessary. The temperature must be high enough that all components are in the liquid state but not so high as to jeopardize the stability of the components. A working temperature range is generally from about 20° C. to about 70° C. To create an oil-in-water emulsion, the final oil phase is gently mixed into either an intermediate, preferably de-ionized water phase, or the final aqueous phase to create a suitable dispersion and the product is allowed to cool with or without stirring. In the case wherein the final oil phase is first gently mixed into an intermediate water phase, this emulsion concentrate is thereafter mixed in the appropriate ratio with the final aqueous phase. The final aqueous phase includes the water soluble polymer as well as other aqueous-soluble components. In such cases, the emulsion concentrate and the final aqueous phase need not be at the same temperature or heated above room temperature, as the emulsion has already been formed at this point.

Semisolids may form in the process of self-emulsification if the amount of ethylene oxide units in one emulsifier is too large. Generally, if the surfactant or surfactants have more than 10 ethylene oxide units in their structures, the surfactant and oil phase is mixed with a small amount of the total composition water, e.g., about 0.1-10%, to first form a semi-solid substance in the form of a milky paste for average droplet sizes of about 0.18 micron and a clear viscous gel for mean droplet sizes of less than about 0.1 micron, which is thereafter combined with the remaining water. Gentle mixing may then be required until the hydrated emulsifiers are fully dissolved to form the emulsion.

In one embodiment, the surfactant and oil are initially combined and heated. A small amount of the aqueous phase is then added to the oil phase to form a semi-solid substance in the form of a milky paste for average droplet sizes of about 0.18 micron and a clear viscous gel for mean droplet sizes of less than 0.1 micron. The amount of the aqueous phase added may be from 0.1 to 10%, preferably from 0.5 to 5% and most preferably 1 to 2%. After the gel is formed, additional water is added to the gel at the same temperature as above. In some embodiments, the amount of water added is 5 to 20%. The emulsion is then gently mixed. In some embodiments, mixing may occur for 30 minutes to 3 hours.

In a preferred embodiment, the particles are then sized. A Horiba LA-920 particle size analyzer may be used according to the manufacturer's instructions for this purpose. In a preferred embodiment, the particles are between 0.08 and 0.18 micron in size before passing to the next step.

In the next step, the particles may be mixed with other aqueous components such as water, one or more demulcents and buffer (preferably boric acid based). Optionally, electrolytes, such as calcium chloride dihydrate, magnesium chloride hexahydrate, potassium chloride and sodium chloride, and Kollidon 17 NF may be added. While the electrolytes are not necessary to form the emulsions, they are very helpful to preserve ocular tissue integrity by maintaining the electrolyte balance in the eye. Likewise, the buffer is not critical, but a boric acid/sodium borate system is preferred in one embodiment of the invention because a phosphate-based buffer system will precipitate with the preferred electrolytes.

The pH is adjusted to 6.5 to 8.5, preferably from about 7.3 to 7.7. This pH range is optimal for tissue maintenance and to avoid ocular irritation. A preservative may then be added. In a preferred embodiment, a polymeric quartenary amine is added. In a preferred embodiment, polyhexamethylene biguanide (PHMB) is added.

The oil-in-water emulsions of the present invention can be sterilized after preparation using autoclave steam sterilization or can be sterile filtered by any means known in the art. Sterilization employing a sterilization filter can be used when the emulsion droplet (or globule or particle) size and characteristics allows. The droplet size distribution of the emulsion need not be entirely below the particle size cutoff of the sterile filtration membrane to be sterile-filtratable. In cases where the droplet size distribution of the emulsion is above the particle size cutoff of the sterile filtration membrane, the emulsion needs to be able to deform or acceptably change while passing through the filtrating membrane and then reform after passing through. This property is easily determined by routine testing of emulsion droplet size distributions and percent of total oil in the compositions before and after filtration. Alternatively, a loss of a small amount of larger droplet-sized material may be acceptable.

The emulsions of the present invention are generally non-aseptically filtered through a clarification filter before sterile filtration or aseptically clarify-filtered after autoclave steam sterilization. In a preferred embodiment, the emulsion is filter sterilized using a 0.22 micron filter. Preferably, 98 to 99% of the emulsion should pass through the 0.22 micron filter. Note that particles larger than 0.22 micron may pass through by altering their shape temporarily. In a preferred embodiment, the material is then tested to verify the effectiveness of the sterilization step. Storage is preferably below 25° C. in order to maintain stability. Thereafter, the emulsions are aseptically filled into appropriate containers. This step is added to sterilize the solution, not to alter droplet size. Droplet size is determined by the amount of surfactant that is added to the solution.

The present invention provides for methods of using ophthalmic compositions, such as the present ophthalmic compositions described elsewhere herein. In one embodiment, the present methods comprise administering a composition of the invention to an eye of a subject, for example, a human or an animal, in an amount and at conditions effective to provide at least one benefit to the eye. In this embodiment, the present composition can employ at least one portion of the composition, for example, a therapeutic component and the like, useful for treating a condition, for example, dry eye and/or one or more other conditions of the eye.

In a useful embodiment, the present methods comprise contacting a contact lens with a composition of the present invention in an amount and at conditions effective to provide at least one benefit to the contact lens and/or the wearer of the contact lens. In this embodiment, the present composition is employed as at least a portion of a contact lens care composition.

Compositions according to the invention may be used in methods which comprise administering the composition to an eye of a subject, that is a human or animal, in an amount effective in providing a desired therapeutic effect to the subject. Such therapeutic effect may be an ophthalmic therapeutic effect and/or a therapeutic effect directed to one or more other parts of the subject's body or systemically to the subject's body. In preferred embodiments, the therapeutic effect is treatment and/or relief from symptoms of dry eye.

The aqueous phase or component and the oil phase and component used in accordance with the present invention are selected to be effective in the present compositions and to have no substantial or significant deleterious effect, for example, on the compositions, on the use of the compositions, on the contact lens being treated, on the wearer of the treated lens, or on the human or animal in whose eye the present composition is placed.

The liquid aqueous medium or component of the present compositions preferably includes a buffer component which is present in an amount effective to maintain the pH of the medium or aqueous component in the desired range. The present compositions preferably include an effective amount of a tonicity adjusting component to provide the compositions with the desired tonicity.

The aqueous phase or component in the present compositions may have a pH which is compatible with the intended use, and is often in the range of about 4 to about 10. A variety of conventional buffers may be employed, such as phosphate, borate, citrate, acetate, histidine, tris, bis-tris and the like and mixtures thereof. Borate buffers include boric acid and its salts, such as sodium or potassium borate. Potassium tetraborate or potassium metaborate, which produce boric acid or a salt of boric acid in solution, may also be employed. Hydrated salts such as sodium borate decahydrate can also be used. Phosphate buffers include phosphoric acid and its salts; for example, M₂HPO₄ and MH₂PO₄, wherein M is an alkali metal such as sodium and potassium. Hydrated salts can also be used. In one embodiment of the present invention, Na₂HPO₄ is used. 7H₂O and NaH₂PO₄.H₂O are used as buffers. The term phosphate also includes compounds that produce phosphoric acid or a salt of phosphoric acid in solution. Additionally, organic counter-ions for the above buffers may also be employed. The concentration of buffer generally varies from about 0.01 to 2.5 w/v % and more preferably varies from about 0.05 to about 0.5 w/v %.

The type and amount of buffer are selected so that the formulation meets the functional performance criteria of the composition, such as surfactant and shelf life stability, antimicrobial efficacy, buffer capacity and the like factors. The buffer is also selected to provide a pH, which is compatible with the eye and any contact lenses with which the composition is intended for use. Generally, a pH close to that of human tears, such as a pH of about 7.45, is very useful, although a wider pH range from about 6 to about 9, more preferably about 6.5 to about 8.5 and still more preferably about 6.8 to about 8.0 is also acceptable. In one embodiment, the present composition has a pH of about 7.0.

The osmolality of the present compositions may be adjusted with tonicity agents to a value which is compatible with the intended use of the compositions. For example, the osmolality of the composition may be adjusted to approximate the osmotic pressure of normal tear fluid, which is equivalent to about 0.9 w/v % of sodium chloride in water. Examples of suitable tonicity adjusting agents include, without limitation, sodium, potassium, calcium and magnesium chloride; dextrose; glycerin; propylene glycol; mannitol; sorbitol and the like and mixtures thereof. In one embodiment, a combination of sodium chloride and potassium chloride are used to adjust the tonicity of the composition.

Tonicity agents are typically used in amounts ranging from about 0.001 to 2.5 w/v %. These amounts have been found to be useful in providing sufficient tonicity for maintaining ocular tissue integrity. Preferably, the tonicity agent(s) will be employed in an amount to provide a final osmotic value of 150 to 450 mOsm/kg, more preferably between about 250 to about 330 mOsm/kg and most preferably between about 270 to about 310 mOsm/kg. The aqueous component of the present compositions more preferably is substantially isotonic or hypotonic (for example, slightly hypotonic, e.g., about 240 mOsm/kg) and/or is ophthalmically acceptable. In one embodiment, the compositions contain about 0.14 w/v % potassium chloride and 0.006 w/v % each of calcium and/or magnesium chloride.

In addition to tonicity and buffer components, the present compositions may include one or more other materials, for example, as described elsewhere herein, in amounts effective for the desired purpose, for example, to treat contact lenses and/or ocular tissues, for example, to provide a beneficial property or properties to contact lenses and/or ocular tissues, contacted with such compositions.

In one embodiment, the compositions include a second therapeutic agent in addition to the water-soluble polymer for treatment of dry eye as illustrated in U.S. application Ser. Nos. 11/098,827, filed Apr. 4, 2005; Ser. No. 10/802,153, filed Mar. 17, 2004; and Ser. No. 10/392,375, filed Mar. 18, 2003.

In another embodiment, the present compositions are useful as multi-purpose care compositions, rigid gas permeable soaking and conditioning solutions, rewetting compositions and cleaning compositions, for example, in-the-eye cleaners, for contact lens care.

All types of contact lenses may be cared for using compositions of the present invention. For example, the contact lenses may be soft, rigid and soft or flexible gas permeable, silicone hydrogel, silicon non-hydrogel and conventional hard contact lenses.

A multi-purpose composition, as used herein, is useful for performing at least two functions, such as cleaning, rinsing, disinfecting, rewetting, lubricating, conditioning, soaking, storing and otherwise treating a contact lens, while the contact lens is out of the eye. Such multi-purpose compositions preferably are also useful for re-wetting and cleaning contact lenses while the lenses are in the eye. Products useful for re-wetting and cleaning contact lenses while the lenses are in the eye are often termed re-wetters or “in-the-eye” cleaners. The term “cleaning” as used herein includes the loosening and/or removal of deposits and other contaminants from a contact lens with or without digital manipulation and with or without an accessory device that agitates the composition. The term “re-wetting” as used herein refers to the addition of water over at least a part, for example, at least a substantial part, of at least the anterior surface of a contact lens.

Although the present compositions are very effective as multi-purpose contact lens care compositions, the present compositions, with suitable chemical make-ups, can be formulated to provide a single contact lens treatment. Such single treatment contact lens care compositions, as well as the multi-purpose contact lens care compositions are included within the scope of the present invention.

Methods for treating a contact lens using the herein described compositions are included within the scope of the invention. In general, such methods comprise contacting a contact lens with such a composition at conditions effective to provide the desired treatment to the contact lens.

The contact lens can be contacted with the composition, often in the form of a liquid aqueous medium, by immersing the lens in the composition. During at least a portion of the contacting, the composition containing the contact lens can be agitated, for example, by shaking the container containing the composition and contact lens, to at least facilitate the contact lens treatment, for example, the removal of deposit material from the lens. Before or after such contacting step, in contact lens cleaning, the contact lens may be manually rubbed to remove further deposit material from the lens. The cleaning method may optionally also include rinsing the lens prior to or after the contacting step and/or rinsing the lens substantially free of the composition prior to returning the lens to the wearer's eye.

In addition, methods of applying or administering artificial tears, washing eyes and irrigating ocular tissue, for example, before, during and/or after surgical procedures, are included within the scope of the present invention. The present compositions, as described elsewhere herein, are useful as artificial tears, eyewash and irrigating compositions which can be used, for example, to replenish/supplement natural tear film, to wash, bathe, flush or rinse the eye following exposure to a foreign entity, such as a chemical material or a foreign body or entity, or to irrigate ocular tissue subject to a surgical procedure. Foreign entities in this context include, without limitation, one or more of pollen, dust, ragweed and other foreign antigens, which cause adverse reactions, such as allergic reactions, redness, itching, burning, irritation, and the like in the eye.

The present compositions, having suitable chemical make-ups, are useful in each of these, and other, in-the-eye applications. These compositions can be used in in-the-eye applications in conventional and well-known manners. In other words, a composition in accordance with the present invention can be used in an in-the-eye application in a substantially similar way as a conventional composition is used in a similar application. One or more of the benefits of the present compositions, as discussed elsewhere herein, are provided as the result of such in-the-eye use.

A cleaning component may be included in the present compositions useful to clean contact lenses as illustrated in U.S. application Ser. Nos. 11/098,827, filed Apr. 4, 2005; Ser. No. 10/802,153, filed Mar. 17, 2004; and Ser. No. 10/392,375, filed Mar. 18, 2003.

The present compositions may further comprise one or more antimicrobial agents (i.e., preservatives or disinfectants) to preserve the compositions from microbial contamination and/or disinfect contact lenses. The amount of the preservative component present in the liquid aqueous medium is effective to disinfect a contact lens placed in contact with the composition.

In one embodiment, for example, when a multi-purpose contact lens composition is desired, the preservative component includes, but is not limited to, a polymeric quaternary amine such as polyhexamethylene biguanide (PHMB), Polyquaternium-1, ophthalmically acceptable salts thereof, and the like and mixtures thereof.

Preservative component selection for the oil-in-water emulsions according to embodiments of the invention can be facilitated by using the HLB (Hydrophile-Lipophile Balance) system. The HLB number of the oil component can be obtained from the supplier or from compiled lists in the literature. The HLB number for simple alcohol ethoxylate surfactants may be readily calculated. HLB values for other ethoxylates may be determined experimentally. Overall chemical structure (e.g., branched, linear, aromatic) is also a variable. HLB values are additive; therefore, if two different surfactants or oils are present, the HLB will be the weighted average of the HLB values for each component. In preferred embodiments of the invention, the HLB for the cationic antimicrobial component is significantly higher than the HLB of the oil component. More preferably, the cationic antimicrobial has an HLB value at least 2 HLB units higher than the HLB value of the oil component. Yet more preferably, the cationic antimicrobial has an HLB value at least 5 HLB units higher than the HLB value of the oil component.

The preservative components useful in the present invention are preferably present in the present compositions in concentrations in the range of about 0.00001% to about 2% (w/v).

In preferred embodiments, PHMB is present at a concentration of about 0.0001% (w/w).

More preferably, the preservative component is present in the present compositions at an ophthalmically acceptable or safe concentration such that the user can remove the disinfected lens from the composition and thereafter directly place the lens in the eye for safe and comfortable wear.

Sufficient amounts of preservative component are used, such that the preservative component reduces the microbial burden on the contact lens by greater than 3 log drops in 7 days for Staphylococcus aureus, Pseudomonas aeruginosa, Escherichia coli and that there is no growth for Candida albicans and Aspergillus niger.

The preservative component is preferably provided in the present composition, and is more preferably soluble in the aqueous component of the present composition.

Other useful preservatives include antimicrobial peptides. Among the antimicrobial peptides which may be employed include, without limitation, defensins, peptides related to defensins, cecropins, peptides related to cecropins, magainins and peptides related to magainins and other amino acid polymers with antibacterial, antifungal and/or antiviral activities. Mixtures of antimicrobial peptides or mixtures of antimicrobial peptides with other preservatives are also included within the scope of the present invention.

The compositions of the present invention may include viscosity modifying agents or components, such as cellulose polymers, including hydroxypropyl methyl cellulose (HPMC), hydroxyethyl cellulose (HEC), ethyl hydroxyethyl cellulose, hydroxypropyl cellulose, methyl cellulose and carboxymethyl cellulose (CMC); carbomers (e.g. carbopol RTM); polyvinyl alcohol; polyvinyl pyrrolidone; alginates; carrageenans; and guar, karaya, agarose, locust bean, tragacanth and xanthan gums. Such viscosity modifying components are employed, if at all, in an amount effective to provide a desired viscosity to the present compositions. The concentration of such viscosity modifiers will typically vary between about 0.01 to about 5% w/v of the total composition, although other concentrations of certain viscosity modifying components may be employed.

The compositions of the present invention may also include viscosity modifying agents such as dextran 70, gelatin, glycerine, polyethylene glycols (e.g., PEG 300 and PEG 400), polysorbate 80, propylene glycol, povidone and the like and mixtures thereof. Such viscosity modifying components are employed, if at all, in an amount effective to provide a desired viscosity to the present compositions. The concentration of such viscosity modifiers will typically vary between about 0.01 to about 5% w/v of the total composition, although other concentrations of certain viscosity modifying components may be employed.

It is desirable in some instances to include sequestering agents or components in the present compositions in order to, and in an amount effective to, bind metal ions, which, for example, might otherwise stabilize cell membranes of microorganisms and thus interfere with optimal disinfection activity. Alternatively, it is desirable in some instances to bind metal ions to prevent their interaction with other species in the compositions. Sequestering agents are included, if at all, in amounts effective to bind at least a portion, for example, at least a major portion of the metal ions present. Such sequestering components usually are present in amounts ranging from about 0.01 to about 0.2 w/v %. Examples of useful sequestering components include, without limitation ethylene-diaminetetraacetic acid (EDTA) and its potassium or sodium salts and low molecular weight organic acids such as citric and tartaric acids and their salts, e.g., sodium salts.

The present compositions may comprise effective amounts of one or more additional components. For example, one or more conditioning components or one or more contact lens wetting agents and the like and mixtures thereof may be included. Acceptable or effective concentrations for these and other additional components in the compositions of the invention are readily apparent to the skilled practitioner.

Each of the components may be present in either a solid or liquid form of the present compositions. When the additional component or components are present as a solid, they can either be intimately admixed such as in a powder or compressed tablet or they can be substantially separated, although in the same particles, as in an encapsulated pellet or tablet. The additional component or components can be in solid form until desired to be used, whereupon they can be dissolved or dispersed in the aqueous component of the present composition in order to, for example, effectively contact the surface of a contact lens.

When any component is included, it is preferably compatible under typical use and storage conditions with the other components of the composition.

In certain embodiments, an antimicrobial activity of the ophthalmic compositions described herein increases after production. Post-production treatment may include storage of the composition for a period of time from one week to several months, preferably two to six weeks, and most preferably, at least about one month post production. The increase in microbial activity may also be enhanced by treatment with heat, pressure or oxidizing conditions. A combination of treatments may be used. For example, the composition may be stored at a temperature of 30-50° C., more preferably, about 40° C. for a period of at least about two weeks, most preferably, one month.

The ophthalmic compositions according to the invention have the following unexpected properties.

1) It was unexpectedly discovered that when mean emulsion droplet sizes small, the emulsions containing Omega-3 fatty acids are stable. For example, if mean emulsion droplet sizes are reduced to less than 0.1 micron in a solution that contains 3% Lumulse GRH-40, 1% flaxseed oil, 0.5% boric acid, 0.035% sodium borate decahydrate, 0.14% KCl, and 0.25% NaCl, the resulting microemulsion is stable in a low viscosity solution at a range concentration of CMC from 0.1% (w/w) to 3.0% (w/w).

2) It was unexpectedly discovered that when polymeric quartenary amines such as PHMB are combined with Omega-3 oil emulsions, their antimicrobial activity is not significantly reduced, such that PHMB still meets the FDA requirements for a preservative and thus can be used as a preservative in Omega-3 fatty acid emulsions and mircoemulsions.

3) It was unexpectedly discovered that polymeric quartenary amines are compatible with Omega-3 oil emulsions while oxidative preservatives and non-polymeric preservatives lose antimicrobial activity when placed in an emulsion containing Omega-3 fatty acids. For example, when PHMB is added to a solution that contains 1.5% Lumulse GRH-40, 1% Perilla oil, 0.6% boric acid, 0.035% sodium borate, 0.14% KCl, and 0.35% NaCl, PHMB does not show any reduced antimicrobial activity after 7 days with Staphylococcus aureus, Pseudomonas aeruginosa and Escherichia coli, while CPC and Alexidine show reduced antimicrobial activity after 7 days in the same solution. When stabilized ClO₂ is placed in a solution that contains 0.8% Lumulse GRH-40, 1% flaxseed oil, 0.0072 stabilized ClO₂, 0.1% boric acid, 0.2% sorbitol, 0.69% 1N NaOH, 0.006% CaCl₂ 2H₂0, 0.006% MgCl₂.6H₂0, 0.14% KCl, and 0.25% NaCl, stabilized ClO₂ levels drop due to interaction with Omega-3 fatty acids.

It will be understood by those of skill in the art that numerous and various modifications can be made without departing from the spirit of the present invention. Therefore, it should be clearly understood that the following examples are illustrative only and are not intended to limit the scope of the present invention.

### EXAMPLES

### Example 1

### Method of Preparing Ophthalmic Solution

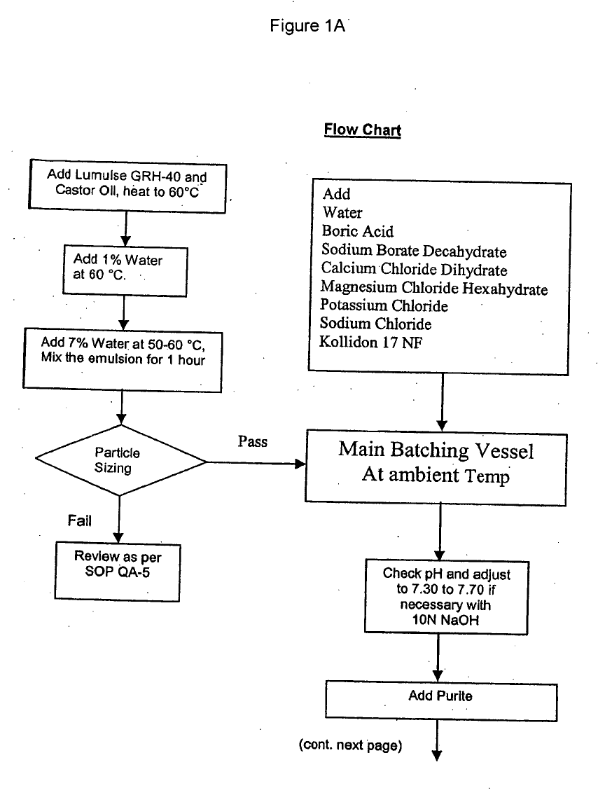

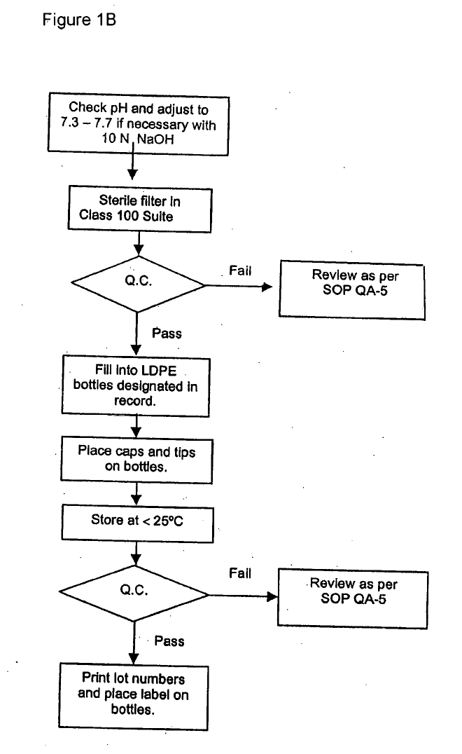

Detailed methods of preparing self-emulsifying compositions may be found in U.S. application Ser. No. 10/802,153, filed Mar. 17, 2004 which is incorporated herein by reference. The following example describes a one-component surfactant system. In this example, PEG-40 hydrogenated castor oil, a 40 mole ethoxylated derivative of hydrogenated castor oil, is exemplified. Reference is made to FIG. 1 and Table 1. FIG. 1 shows a flow chart for the method. Table 1 shows amounts of the various components for this example.

PEG-40 hydrogenated castor oil (Lumulse GRH-40, Lambent Technologies Corp., Skokie, Ill.) and castor oil were heated. The temperature must be high enough that all components are in the liquid state but not so high as to jeopardize the stability of the components. In the present example, a temperature of 60+/−2° C. was used.

A small amount of the total water (1%) was added at 60+/−2° C., to form a transparent white paste. The paste was mixed until the mixture was homogenous. After the paste was formed, more water was added to the paste between 50-62° C. In this example, 7% of the total water was added and mixing was carried out for 1 hour at 200-1000 rpm until the mixture was homogeneous. At this stage, an emulsion concentrate had formed.

The particles (droplets) were then sized using a Horiba LA-920 particle size analyzer according to the manufacturer's instructions. Particles which were between 0.08 and 0.18 micron in size were allowed to pass to the next step.

The emulsion concentrate was mixed with a separately prepared solution of the remaining water, buffer, electrolytes (calcium chloride dihydrate, magnesium chloride hexahydrate, potassium chloride and sodium chloride) and Kollidon 17 NF (see Table 1) for about 30 minutes. While the electrolytes are not necessary to form the emulsions, they are very helpful to preserve ocular tissue integrity by maintaining the electrolyte balance in the eye. Likewise, the buffer is not critical to form the emulsion, but is necessary to properly maintain a compatible ocular pH. A boric acid/sodium borate buffer system is preferred because a phosphate-based buffer system will precipitate with the electrolytes. Water soluble polymers such as demulcents for the treatment of dry eye may be added at this stage to form other embodiments of the present invention.

The pH was adjusted to 7.35 to 7.55 with 10N NaOH. This pH range is optimal for tissue maintenance and to avoid ocular irritation and is the optimal pH range for stability of Purogene® which was added as a preservative. Purogene® was added according to the calculation shown in Table 1. Thereafter, pH was checked and adjusted to pH 7.5+/−0.2 with 10N NaOH. Note that the pH may only be adjusted with a base such as 10 N NaOH after the addition of Purogene®, as high local solution concentrations of acid formed during acid pH adjustment will cause destruction of the Purogene®.

In the next step, the emulsion was stored covered in the dark at less than 25° C. until sterile filtered. Maximum storage time is 72 hours.

The composition was then filter sterilized using a 0.22 micron filter. 98-99% of the emulsion passed through the 0.22 micron filter. Note that particles larger than 0.22 micron may pass through by altering their shape temporarily. The material was then tested to verify the effectiveness of the sterilization step. The material was then bottled and stored. Pre-fill release specifications for this example were pH 7.3-7.7, mean particle size of 0.09-0.17 micron and physical appearance of a milky white solution. Post-fill release specifications were pH 7.3-7.7, potential chlorine dioxide of 60-70 ppm, castor oil 1.1-1.4% (w/w), Kollidon 17 NF 0.2-0.4% (w/w), osmolality 250-280 mOsm/kg, and sterility USP.

TABLE 1
Emulsion formulation for example 1

| Ingredient/Component | Amount/1000 g |
|---|---|
| Lumulse GRH-40 | 10 |
| Castor oil | 12.5 |
| Boric Acid | 6.0 |
| Sodium Borate | 0.35 |
| Calcium Chloride dihydrate | 0.06 |
| Magnesium Chloride hexahydrate | 0.06 |
| Potassium Chloride | 1.4 |
| Sodium Chloride | 3.5 |
| Kollidon 17 PF | 3.0 |
| 10 N Sodium Hydroxide | pH adjust |
| Purogene ® | see below¹ |
| Purified Water, USP | see below² |
| Sterile filter, 0.22 micron | |

¹Purogene ® calculation: the amount of raw material to be added must be calculated on the basis of the assay of the raw material lot. 0.0065% (w/w) × 1000 g = grams of Purogene ® raw material Purogene ® raw material assay value % (w/w) required per 1000 g Purogene ® (g) required per 1000 g/1000 g × Batch size (g) = Purogene ® (g) required/batch size
²Water amount calculation per 1000 g The amount of water to be added must be calculated on the basis of the amount of Purogene ® raw material to be added. Water (g) per 1000 g = 963.13 − Purogene ® (g) required per 1000 g

### Example 2

### Characterization of Emulsions Containing HA

Empirical data has shown that hyaluronic acid in certain concentrations can destabilize the emulsion, so as to cause creaming. Examples 2 and 3 illustrate stable and unstable combinations (designation of “unstable” indicates that creaming was observed) with the emulsion formulation and sodium hyaluronate. The formulations in the following examples were prepared essentially as described in Example 1.

TABLE 2
Emulsion formulations for Example 2.

| Ingredients | % w/w | % w/w | % w/w | % w/w |
|---|---|---|---|---|
| Sodium hyaluronate | 0.1 | 0.2 | 0.3 | 0.4 |
| Castor Oil | 1.25 | 1.25 | 1.25 | 1.25 |
| POE(40) Hydrogenated Castor | 1 | 1 | 1 | 1 |
| Oil | | | | |
| Sodium Chlorite | 65 ppm | 65 ppm | 65 ppm | 65 ppm |
| WSCP | 3 ppm | 3 ppm | 3 ppm | 3 ppm |
| Boric Acid | 0.6 | 0.6 | 0.6 | 0.6 |
| Sodium Borate Decahydrate | 0.035 | 0.035 | 0.035 | 0.035 |
| Calcium chloride dihydrate | 0.006 | 0.006 | 0.006 | 0.006 |
| Magnesium chloride | 0.006 | 0.006 | 0.006 | 0.006 |
| hexahydrate | | | | |
| Potassium chloride | 0.14 | 0.14 | 0.14 | 0.14 |
| Sodium chloride | 0.35 | 0.35 | 0.35 | 0.35 |
| Purified water | QS | QS | QS | QS |
| Emulsion stability | Stable | Stable | Unstable | Unstable |

Table 2 above shows that stable oil-in-water emulsions were obtained when the HA concentration was 0.2 w/w % or less.

### Example 3

### Incorporation of HA to form a Stable Emulsion System when the HA Concentration is Low

TABLE 3
Emulsion formulations for Example 3.

| Ingredients | % w/w | % w/w | % w/w | % w/w | % w/w |
|---|---|---|---|---|---|
| Sodium | 0.05 | 0.2 | 0.3 | 0.5 | 0.7 |
| Hyaluronate | | | | | |
| Castor oil | 0.313 | 0.313 | 0.313 | 0.313 | 0.313 |
| Lumulse GRH-40 | 0.25 | 0.25 | 0.25 | 0.25 | 0.25 |
| PHMB (ppm) | 1 ppm | 1 ppm | 1 ppm | 1 ppm | 1 ppm |
| Dibasic sodium | 0.12 | 0.12 | 0.12 | 0.12 | 0.12 |
| phosphate (7H₂O) | | | | | |
| Monobasic | 0.01 | 0.01 | 0.01 | 0.01 | 0.01 |
| sodium phosphate | | | | | |
| (H₂O) | | | | | |
| Edetate disodium | 0.01 | 0.01 | 0.01 | 0.01 | 0.01 |
| Taurine | 0.05 | 0.05 | 0.05 | 0.05 | 0.05 |
| Potassium | 0.14 | 0.14 | 0.14 | 0.14 | 0.14 |
| chloride | | | | | |
| Sodium chloride | 0.75 | 0.75 | 0.75 | 0.75 | 0.75 |
| Purified water | QS | QS | QS | QS | QS |
| Emulsion stability | Stable | Stable | Unstable | Unstable | Unstable |

Table 3 shows that stable oil-in-water emulsions were obtained when the HA concentration is 0.2 w/w % or less, even when the emulsion concentration is lowered to one fourth of the concentration of Example 2 (Table 2).

### Example 4

Example 4 illustrates that when the HA concentration was maintained constant at 0.2% w/w, but the emulsion concentration was lowered further to ⅛× concentration, the emulsion/HA compositions became unstable.

TABLE 4
Emulsion formulations for Example 4.

| | ⅛X | ¼X | 1X |
|---|---|---|---|
| Ingredients | % w/w | % w/w | % w/w |
| Sodium Hyaluronate | 0.2 | 0.2 | 0.2 |
| Castor oil | 0.156 | 0.313 | 1.25 |
| Lumulse GRH-40 | 0.125 | 0.25 | 1 |
| Sodium chlorite | | | 65 ppm |
| WSCP | | | 3 ppm |
| Boric Acid | | | 0.6 |
| Sodium borate decahydrate | | | 0.035 |
| Calcium chloride dihydrate | | | 0.006 |
| Magnesium chloride hexahydrate | | | 0.006 |
| PHMB (ppm) | 1 ppm | 1 ppm | |
| Dibasic sodium phosphate (7H₂O) | 0.12 | 0.12 | |
| Monobasic sodium phosphate (H₂O) | 0.01 | 0.01 | |
| Edetate disodium | 0.01 | 0.01 | |
| Taurine | 0.05 | 0.05 | |
| Potassium chloride | 0.14 | 0.14 | 0.14 |
| Sodium chloride | 0.75 | 0.75 | 0.35 |
| Purified water | QS | QS | QS |
| Emulsion stability | Unstable | Stable | Stable |

The above examples illustrate that when the HA concentration is too high or when the emulsion concentration is not sufficient, the HA/Emulsion combination is unstable. However, stable HA/Emulsion compositions were obtained at HA concentrations of at least 0.2% w/w and emulsion concentrations which are equal to or greater than ¼×. While these examples are shown for HA, stable formulations for other water-soluble polymer demulcents may be determined similarly.

### Example 5

### Effect of Surfactant on Quaternary-Based Antimicrobial Activity

FDA/ISO specified test organisms are listed below:
Serratia marcescens, ATCC 13880
Staphylococcus aureus, ATCC 6538
Pseudomonas aeruginosa, ATCC 9027
Candida albicans, ATCC 10231
Fusarium solani, ATCC 36031

(FDA Premarket Notification (510 k) Guidance Document for Contact Lens Care Products, Appendix B, Apr. 1, 1997 and ISO/FDIS 14729: Ophthalmic optics—Contact lens care products—Microbiological requirements and test methods for products and regimens for hygienic management of contact lenses, January 2001). Contact lens disinfectants are also known as contact lens multi-purpose solutions, when they are used for rinsing, cleaning, disinfection, storage and rewetting contact lenses.

FDA and ISO guidelines specify two disinfection efficacy standards, defined in Table 5 below. Disinfectants are directly challenged with Pseudomonas aeruginosa, Staphylococcus aureus, Serratia marcescens, Candida albicans, and Fusarium solani. The primary criteria for passing state that a minimum 99.9% (3.0 logs) reduction is required for each of the three bacterial types within the minimum recommended soaking period. Mold and Yeast must meet a minimum 90.0% (1.0 log) reduction within the minimum recommended soaking period with no increase (stasis) at not less than four times the minimum recommended soaking period within an experimental error of ±0.5 logs. If the primary criteria is met, the composition may be labeled as a disinfectant.

If the primary criteria is not met the secondary criteria states that the sum of the averages must be a minimum of 5.0 log units reduction for the three species of bacteria within the recommended soaking period with a minimum average of 1.0 log unit reduction for any single bacteria. Stasis for the yeast and mold shall be observed for the recommended soaking period within an experimental error of ±0.5 logs. The composition may be labeled as part of a disinfectant regiment if it passes the second criteria.

TABLE 5
Disinfection efficacy standards.

| | |
|---|---|
| Organism | Average log reduction at labeled soak time |

Stand Alone Disinfectant (Primary) Criteria:

| | |
|---|---|
| S. marcescens | 3.0 logs |
| S. aureus | 3.0 logs |
| P. aeruginosa | 3.0 logs |
| C. albicans | 1.0 log |
| F. solani | 1.0 log |

Regimen-Dependent Disinfectant (Secondary) Criteria:

| | |
|---|---|
| S. marcescens | Minimum of 1.0 log per bacterium, |
| S. aureus | sum of all three bacteria log-drops |
| P. aeruginosa | must be greater than or equal to 5.0 log |
| C. albicans | Stasis |
| F. solani | Stasis |

Antimicrobial activity provided by quaternary-based antimicrobials is frequently lost in the presence of a large amount of surfactant containing alkyl chains, such as POE(40) Hydrogenated Castor Oil. In fact, Tween 80 is routinely used as a quaternary ammonium neutralizer in antimicrobial activity testing. The surfactant forms micelles, which strongly adsorb the antimicrobial, thereby reducing the activity. Table 6 below shows that the alkyl groups in the emulsion can also adsorb the quaternary ammonium molecules thereby inactivating antimicrobial activity.

TABLE 6
Effect of emulsion on log drop

| | | CPC | Alexidine | Alexidine |
|---|---|---|---|---|
| | CPC with | without | with | without |
| Ingredients | emulsion | emulsion | emulsion | emulsion |
| Castor Oil | 0.625 | | 0.625 | |
| Lumulse GRH-40 | 0.500 | | 0.500 | |
| Sodium Hyaluronate | 0.1 | 0.05 | | 0.5 |
| PVP | | | 0.15 | |
| Cetylpyridinium Chloride | 5 ppm | 2 ppm | | |
| Alexidine | | | 2.5 ppm | 2 ppm |
| Tris HCl | | 0.055 | | 0.055 |
| Tris base | | 0.021 | | 0.021 |
| Pluronic F87 | | 0.05 | | 0.05 |
| Propylene glycol | | 0.5 | | 0.5 |
| Dibasic Sodium | 0.12 | | 0.12 | |
| Phosphate (7H₂O) | | | | |
| Monobasic Sodium | 0.01 | | 0.01 | |
| Phosphate (1H₂O) | | | | |
| Taurine | 0.05 | 0.05 | 0.05 | 0.05 |
| Potassium Chloride | 0.14 | 0.14 | 0.14 | 0.14 |
| Sodium Chloride | 0.75 | 0.59 | 0.75 | 0.59 |
| Edetate Disodium | 0.01 | 0.01 | 0.01 | 0.01 |
| Purified water | QS | QS | QS | QS |

LOG DROP AT 6 HOURS

| | | | | |
|---|---|---|---|---|
| S. marcescens ATCC | 0.81 | 4.1 | 0.41 | 4.9 |
| 13880 | | | | |
| S. aureus ATCC 6538 | 0.15 | 3.98 | 0.35 | 3.3 |
| P. aeruginosa ATCC | 0.31 | 4.56 | 1.52 | 4.6 |
| 9027 | | | | |
| C. albicans ATCC | −0.13 | 2.8 | 0.14 | 1.7 |
| 10231 | | | | |
| F. solani ATCC 36031 | 0.15 | 2.44 | 0.25 | 2.9 |
| Sum | 1.3 | 17.9 | 2.7 | 17.4 |

As can be seen from Table 6, the log drop in the presence of the surfactant Lumulse GRH-40 is much lower than in the absence of the surfactant. Loss of antimicrobial activity is a problem for ophthalmic compositions. This problem is solved by the ophthalmic compositions according to the invention. These ophthalmic compositions retain antimicrobial activity even in the presence of surfactant as shown below.

### Example 6

### Incorporation of Quaternary Ammonium Antimicrobial into the Emulsion Formulation

The formulation of Table 7 was prepared as described in Example 1. Antimicrobial testing is shown in Table 8.

TABLE 7
WSCP System with Emulsion

| Ingredients | % W/W |
|---|---|
| Castor oil | 0.625 |
| Lumulse GRH-40 | 0.5 |
| Sodium hyaluronate | 0.2 |
| Boric Acid | 0.6 |
| Sodium Borate Decahydrate | 0.03 |
| Calcium chloride dihydrate | 0.006 |
| Magnesium chloride hexahydrate | 0.006 |
| Potassium chloride | 0.14 |
| Sodium chloride | 0.35 |
| Final Volume | 100 |
| pH | 7.5 |
| Sodium chlorite | 65 ppm |
| WSCP | 0.5 ppm |

TABLE 8
Log drops for the Formulation of Table 7
Log Drops

| | | | 7 | 14 | 21 | 28 |
|---|---|---|---|---|---|---|
| Organism | 6 hrs | 24 hrs | days | days | days | days |
| S. aureus ATCC 6538 | 0.5 | 2.4 | 4.8 | 4.8 | 3.9 | 3.9 |
| P. aeruginosa ATCC 9027 | 0.5 | 4.3 | 4.7 | 4.7 | 3.6 | 3.6 |
| E. coli ATCC 8739 | 0.7 | 4.5 | 4.5 | 4.5 | 3.9 | 3.9 |
| C. albicans ATCC 10231 | | | 3.7 | 4.7 | 3.5 | 3.5 |
| A. niger ATCC 16404 | | | 1.0 | 1.0 | 0.4 | 0.5 |

Surprisingly, the antimicrobial activity increases with aging of the HA-containing emulsions and by 7 days, the criteria for primary disinfectant is met. Furthermore, the criteria for preservative efficacy testing as defined below (Table 9) is also met.

TABLE 9
Preservative Efficacy Testing Criteria

| Organism | USP/FDA/ISO: | European Standards |
|---|---|---|
| S. aureus ATCC 6538 | 1.0 log at 7 days | 1.0 log at 24 hours |
| P. aeruginosa ATCC 9027 | 3.0 logs at 14 days | 3.0 log at 7 days |
| E. coli ATCC 8739 | (rechallenge at 14 | (rechallenge at 14 |
| | days) | days) |
| | no increase at 28 days | no increase at |
| | | 28 days |
| C. albicans ATCC 10231 | Stasis | Stasis |
| A. niger ATCC 16404 | | |

### Example 7

### PHMB in HA/Emulsion System

This example shows the HA/Emulsion system with PHMB as the disinfectant. The composition was prepared with the Formulation of Table 10, essentially as described in Example 1. As can be seen by the results of Table 11, at least the secondary regimen-dependent criteria are met by this formulation.

TABLE 10
Formulation for Example 7.

| Ingredients | % W/W |
|---|---|
| Castor oil | 0.625 |
| PEG (40) Hydrogenated Castor Oil | 0.5 |
| Sodium hyaluronate | 0.1 |
| PHMB | 1 ppm |
| Dibasic sodium phosphate (7H₂O) | 0.12 |
| Monobasic sodium phosphate (1H₂O) | 0.01 |
| Taurine | 0.05 |
| Potassium chloride | 0.14 |
| Sodium chloride | 0.75 |
| Edetate disodium | 0.01 |
| Purified water | QS |
| Sodium hydroxide (pH adjust) | pH 7.2 |

TABLE 11
Log drop at 6 hours for Formulation of Table 10.

| | Log Drop at 6 |
|---|---|
| Organism | hours |
| S. marcescens ATCC 13880 | 3.77 |
| S. aureus ATCC 6538 | 3.62 |
| P. aeruginosa ATCC 9027 | 4.49 |
| C. albicans ATCC 10231 | 0.33 |
| F. solani ATCC 36031 | 2.76 |

### Example 8

### General Description of the Stable Ophthalmic oil-in-Water EMULSIONS WITH OMEGA-3 FATTY ACIDS

The formulations in the following examples were prepared essentially as described in Example 1. Table 12 shows the general description of a stable ophthalmic oil-in-water emulsion that contains Omega-3 fatty acids.

TABLE 12

| Product | |
|---|---|
| formula | % w/w |
| lumulse GRH- | 0.3 |
| 40 | |
| flax seed oil | 0.1 |
| CMC | 1 |
| Taurine | 0.05 |
| Boric acid | 0.6 |
| Sodium borate | 0.07 |
| NaCl | 0.26 |
| KCl | 0.14 |
| PHMB | 0.00008 |
| (preservative) | |

### Example 9

### High Viscosity can Cause Emulsion Instability

Tables 13 through 15 show solutions that contain 1.5% Lumulse GRH-40, 1% flaxseed oil, 0.6% boric acid, 0.035% sodium borate decahydrate, 0.14% KCL, 0.25% NaCl. The mean emulsion sizes are about 0.18 micron.

TABLE 13

| Low viscosity CMC % w/w | Emulsion Stability |
|---|---|
| 0.1 | Stable* |
| 0.2 | Stable |
| 0.3 | Stable |
| 0.4 | Stable |
| 0.5 | Not stable, creamed in 3 days |
| 0.6 | Not stable, creamed overnight |
| 1 | Not stable, creamed overnight |
| 2 | Not stable, creamed overnight |
| 3 | Not stable, creamed overnight |

*“Stable” means the solution can remain homogeneous at least for 6 months.

1) Table 13: In a solution that contains low viscosity CMC, the solution starts to become unstable at a concentration of 0.5% w/w CMC.

TABLE 14

| Medium viscosity CMC % | |
|---|---|
| w/w | Emulsion Stability |
| 0.1 | Stable |
| 0.2 | Stable for 3 months |
| 0.4 | Not stable, creamed in 3 days |
| 0.6 | Not stable, creamed overnight |
| 1 | Not stable, creamed overnight |
| 1.6 | Not stable, creamed overnight |
| 3 | Not stable, creamed overnight |

2) Table 14: In a solution that contains medium viscosity CMC, the solution starts to become unstable at a concentration of 0.4% w/w CMC.

TABLE 15

| High viscosity CMC % w/w | Emulsion Stability |
|---|---|
| 0.2 | White precipitates at 3 months |
| 0.3 | Not stable, creamed in 6 days |
| 0.5 | Not stable, creamed in 6 days |
| 0.6 | Not stable, creamed in 13 days |
| 0.72 | Not stable, creamed in 20 days |

3) Table 15: In a solution that contains high viscosity CMC, the solution starts to become unstable at a concentration of 0.2% w/w CMC.

TABLE 16

| 800K Sodium Hyaluronate | |
|---|---|
| % w/w | Emulsion Stability |
| 0.025 | Stable |
| 0.05 | Stable |
| 0.08 | Stable |
| 0.1 | Stable |
| 0.25 | Not stable, precipitated in 3 |
| | days |
| 0.4 | Not stable, precipitated in 3 |
| | days |
| 0.6 | Not stable, precipitated in 3 |
| | days |
| 1 | Not stable, precipitated in 3 |
| | days |
| 1.2 | Not stable, precipitated after 3 |
| | days |
| 1.5 | Not stable, precipitated after 3 |
| | days |

4) Table 16: In a solution of 800K Sodium Hyaluronate, the solution becomes unstable at a concentration of 0.25% w/w Sodium Hyaluronate.

### Example 10

### High Water Soluble Polymer Concentration can Cause Emulsion Instability

In a solution that contains 1500K Sodium Hyaluronate, 1.5% Lumulse GRH-40, 1% flaxseed oil, 0.6% boric acid, 0.2% Sorbitol, 0.69% sodium hydroxide, 0.14% KCL, 0.25% NaCl and 0.012% sodium chlorite, and mean emulsion sizes about 0.18 micron, the emulsion becomes unstable at a concentration of 0.25% w/w Sodium Hyaluronate.

TABLE 17

| 1500K Sodium Hyaluronate | |
|---|---|
| % w/w | Emulsion Stability |
| 0.025 | Stable |
| 0.050 | Stable |
| 0.075 | Stable |
| 0.1 | Stable |
| 0.25 | Not stable, precipitated in 3 |
| | days |
| 0.4 | Not stable, precipitated in 3 |
| | days |
| 0.6 | Not stable, precipitated in 1 |
| | month |
| 0.8 | Not stable, precipitated in 1 |
| | month |

Stable Emulsions with Omega-3 Fatty Acids

Applicants have surprisingly discovered that despite the difficulties observed in formulating stable emulsions shown in the preceding examples, proper formulation can produce stable emulsions. These stable emulsions are particularly useful when used in connection with pharmaceutical preparations for direct application to the eye, especially for treatment of dry eye.

In the following example, one approach to formulating stable Omega-3-containing emulsions is illustrated, in which the droplet size in the emulsions is kept below 0.1 micron.

### Example 11

### When Mean Emulsion Sizes are Reduced to Less than 0.1 Micron, the Emulsion is Stable Even in High Viscosity

Table 18: In a solution that contains 3% Lumulse GRH-40, 1% flaxseed oil, 0.6% boric acid, 0.035% sodium borate decahydrate, 0.14% KCL, 0.25% NaCl, and mean emulsion sizes less than 0.1 micron, the emulsion is stable in a low viscosity solution at CMC concentrations from 0.1% w/w CMC up to 3% w/w CMC.

TABLE 18

| Low viscosity CMC % w/w | Emulsion Stability |
|---|---|
| 0.1 | Stable |
| 0.2 | Stable |
| 0.3 | Stable |
| 0.4 | Stable |
| 0.5 | Stable |
| 0.6 | Stable |
| 1 | Stable |
| 2 | Stable |
| 3 | Stable |

Thus, even when viscosity is increased through the use of CMC concentrations as high as 3%, stable emulsions can be formed when droplet sizes are kept at 0.1 micron or below.

### Example 12

### Polymeric Quartenary Amine Preservatives are Compatible with Omega-3 Oil Emulsions

Table 19 illustrates a formulation of an Omega-3 oil emulsion using PHMB as a preservative.

TABLE 19

| | % w/w |
|---|---|
| PHMB, | 0.5 |
| ppm | |
| lumulse | 0.6 |
| flaxseed | 0.2 |
| oil | |
| CMC | 1 |
| Taurine | 0.05 |
| Boric acid | 0.6 |
| Sodium | 0.07 |
| borate | |
| NaCl | 0.35 |
| KCl | 0.14 |

Omega-3 oil emulsions would be expected to neutralize polymeric quartenary amines such as PHMB. It was unexpectedly discovered, however, that this does not occur. If PHMB is added to a solution that contains Omega-3 fatty acids, PHMB maintains its antimicrobial activity such that it meets the U.S. and the European preservative efficacy testing criteria shown in Table 9.

Table 20 shows that in a solution that contains 0.5% PHMB, 0.6% Lumulse GRH-40, 0.2% flaxseed oil, 0.6% boric acid, 1% CMC, 0.05% Taurine, 0.6% boric acid, 0.07% sodium borate, 0.25% NaCl and 0.14% KCL (as shown in Table 19), the following log drops occur:

TABLE 20

| | |
|---|---|
| Preservative | Log |
| efficacy | drop |

A - 7 days

| | |
|---|---|
| Sa, 7 d | >4.92 |
| Pa, 7 d | >4.89 |
| Ec, 7 d | 3.34 |
| Ca, 7 d | 1.67 |
| An, 7 d | 2.40 |

B - 14 days

| | |
|---|---|
| Sa, 14 d | >4.92 |
| Pa, 14 d | >4.89 |
| Ec, 14 d | >4.87 |
| Ca, 14 d | 4.14 |
| An, 14 d | 2.45 |

C - 21 days

| | |
|---|---|
| Sa, 21 d | 3.9 |
| Pa, 21 d | 3.9 |
| Ec, 21 d | 3.8 |
| Ca, 21 d | 3.2 |
| An, 21 d | 1.4 |

D - 28 days

| | |
|---|---|
| Sa, 28 d | 3.9 |
| Pa, 28 d | 3.9 |
| Ec, 28 d | 3.8 |
| Ca, 28 d | 3.7 |
| An, 28 d | 1.4 |

A: The log drops after 7 days are greater than 4.92 for Staphylococcus aureus, greater than 4.89 for Pseudomonas aeruginosa, 3.34 for Escherichia coli, 1.67 for Candida albicans and 2.40 for Aspergillus niger.

B: The log drops after 14 days are greater than 4.92 for Staphylococcus aureus, greater than 4.89 for Pseudomonas aeruginosa, greater than 4.87 for Escherichia coli, 4.14 for Candida albicans and 2.45 for Aspergillus niger.

C: The log drops after 21 days are 3.9 for Staphylococcus aureus, 3.9 for Pseudomonas aeruginosa, 3.8 for Escherichia coli, 3.2 for Candida albicans and 1.4 for Aspergillus niger.

D: The log drops after 28 days are 3.9 for Staphylococcus aureus, 3.9 for Pseudomonas aeruginosa, 3.8 for Escherichia coli, 3.7 for Candida albicans and 1.4 for Aspergillus niger.

As can be seen from table 20, the log drops for Staphylococcus aureus, Pseudomonas aeruginosa, Escherichia coli are above the required 3 logs. The logs drops for Candida albicans and Aspergillus niger are also above the required 0 log drop.

### Example 13

### Polymeric Quaternary Amines Preservatives are Compatible with Omega-3 Fatty Acid Emulsions

Table 21: When non-polymeric quaternary amines such as CPC and Alexidine are added to a solution containing Omega-3 fatty acids, they lose their antimicrobial activity. Polymeric quaternary amines such as PHMB do not lose their antimicrobial activity. Table 22 shows the log drops for Staphylococcus aureus, Pseudomonas aeruginosa, and Escherichia coli after 7 days.

TABLE 21

| | A | B | C | D | E | F | G | H |
|---|---|---|---|---|---|---|---|---|
| | % w/w | % w/w | % w/w | % w/w | % w/w | % w/w | % w/w | % w/w |
| CPC | 0.0002 | 0.0002 | 0.001 | | | | | |
| Alexidine | | | | 0.0002 | 0.0002 | 0.001 | | |
| PHMB | | | | | | | 0.0001 | 0.0001 |
| GRH-40 | | 1.5 | 1.5 | | 1.5 | 1.5 | | 1.5 |
| Perilla oil | | 1 | 1 | | 1 | 1 | | 1 |
| (another | | | | | | | | |
| omega-3 oil) | | | | | | | | |
| Boric Acid | 0.6 | 0.6 | 0.6 | 0.6 | 0.6 | 0.6 | 0.6 | 0.6 |
| Sodium | 0.035 | 0.035 | 0.035 | 0.035 | 0.035 | 0.035 | 0.035 | 0.035 |
| Borate | | | | | | | | |
| KCl | 0.14 | 0.14 | 0.14 | 0.14 | 0.14 | 0.14 | 0.14 | 0.14 |
| NaCl | 0.350 | 0.350 | 0.350 | 0.350 | 0.350 | 0.350 | 0.350 | 0.350 |

TABLE 22
Log drop at 7 days.

| | | | | | | | | |
|---|---|---|---|---|---|---|---|---|
| Sa, 7 d | 4.88 | 3.47 | 3.98 | 4.88 | 0.47 | 2.13 | 4.88 | 4.88 |
| Pa, 7 d | 4.89 | 0.65 | 0.52 | 4.89 | 1.22 | 2.39 | 4.89 | 4.89 |
| Ec, 7 d | 4.88 | 0.47 | 0.59 | 4.88 | 2.34 | 4.88 | 4.88 | 4.88 |

A: In a solution that contains 0.0002% (w/w) CPC, 0.6% boric acid, 0.035% sodium borate, 0.14% KCl, and 0.35% NaCl, there is no log drop of antimicrobial activity after 7 days with Staphylococcus aureus, Pseudomonas aeruginosa, and Escherichia coli. The log drop remains at 4.88 with Staphylococcus aureus, 4.89 with Pseudomonas aeruginosa, and 4.88 with Escherichia coli.

B: In a solution that contains 0.0002% (w/w) CPC, 1.5% Lumulse GRH-40, 1% (w/w) Perilla oil, 0.6% boric acid, 0.035% sodium borate, 0.14% KCl, and 0.35% NaCl, there is a log drop of antimicrobial activity after 7 days with Staphylococcus aureus, Pseudomonas aeruginosa, and Escherichia coli. The log drop is 3.47 for Staphylococcus aureus, 0.65 for Pseudomonas aeruginosa, and 0.47 for Escherichia coli.

C: In a solution that contains 0.001% (w/w) CPC, 1.5% Lumulse GRH-40, 1% (w/w) Perilla oil, 0.6% boric acid, 0.035% sodium borate, 0.14% KCl, and 0.35% NaCl, there is a log drop of antimicrobial activity after 7 days with Staphylococcus aureus, Pseudomonas aeruginosa, and Escherichia coli. The log drop is 3.98 for Staphylococcus aureus, 0.52 for Pseudomonas aeruginosa, and 0.59 for Escherichia coli.

D: In a solution that contains 0.0002% (w/w) Alexidine, 0.6% boric acid, 0.035% sodium borate, 0.14% KCl, and 0.35% NaCl, there is no log drop of antimicrobial activity after 7 days with Staphylococcus aureus, Pseudomonas aeruginosa, and Escherichia coli. The log drop remains at 4.88 with Staphylococcus aureus, 4.89 with Pseudomonas aeruginosa, and 4.88 with Escherichia coli.

E: In a solution that contains 0.0002% (w/w) Alexidine, 1.5% Lumulse GRH-40, 1% (w/w) Perilla oil, 0.6% boric acid, 0.035% sodium borate, 0.14% KCl, and 0.35% NaCl, there is a log drop of antimicrobial activity after 7 days with Staphylococcus aureus, Pseudomonas aeruginosa, and Escherichia coli. The log drop is 0.47 for Staphylococcus aureus, 1.22 for Pseudomonas aeruginosa, and 2.34 for Escherichia coli.

F: In a solution that contains 0.001% (w/w) Alexidine, 1.5% Lumulse GRH-40, 1% (w/w) Perilla oil, 0.6% boric acid, 0.035% sodium borate, 0.14% KCl, and 0.35% NaCl, there is a log drop of antimicrobial activity after 7 days with Staphylococcus aureus and Pseudomonas aeruginosa only. The log drop is 2.13 for Staphylococcus aureus, 2.39 for Pseudomonas aeruginosa, and 4.88 for Escherichia coli.

G: In a solution that contains 0.0001% (w/w) PHMB, 0.6% boric acid, 0.035% sodium borate, 0.14% KCl, and 0.35% NaCl, there is no log drop of antimicrobial activity after 7 days with Staphylococcus aureus, Pseudomonas aeruginosa, and Escherichia coli. The log drop remains at 4.88 with Staphylococcus aureus, 4.89 with Pseudomonas aeruginosa, and 4.88 with Escherichia coli.

H: In a solution that contains 0.0001% (w/w) PHMB, 1.5% Lumulse GRH-40, 1% (w/w) Perilla oil, 0.6% boric acid, 0.035% sodium borate, 0.14% KCl, and 0.35% NaCl, there is no log drop of antimicrobial activity after 7 days with Staphylococcus aureus, Pseudomonas aeruginosa, and Escherichia coli. The log drop remains at 4.88 with Staphylococcus aureus, 4.89 with Pseudomonas aeruginosa, and 4.88 with Escherichia coli.

### Example 14

### Oxidative Preservatives are not Compatible with Omega-3 Oil Emulsions

ClO₂ is deceased due to an interaction with Omega-3 fatty acids. Tables 23 and 24 show that initial stabilized ClO₂ levels of 72 ppm decrease due to an interaction with Omega-3 fatty acids. As lumulse levels are increased, lumulse forms a denser coating around the Omega-3 oil droplets, which separates the ClO₂ more effectively from the Omega-3 oil. Therefore, as shown in Tables 23 and 24, there is decreased reduction in ClO₂ levels with increased concentrations of lumulse.

TABLE 23

| | % w/w | % w/w | % w/w | % w/w |
|---|---|---|---|---|
| Lumulse | 0.8 | 1 | 1.2 | 1.5 |
| Flax oil | 1 | 1 | 1 | 1 |
| Stabilized ClO₂ | 0.0072 | 0.0072 | 0.0072 | 0.0072 |
| Boric Acid | 0.1 | 0.1 | 0.1 | 0.1 |
| Sorbitol | 0.2 | 0.2 | 0.2 | 0.2 |
| 1 N NaOH | 0.69 | 0.69 | 0.69 | 0.69 |
| CaCl₂ 2H₂O | 0.006 | 0.006 | 0.006 | 0.006 |
| MgCl₂ 6H₂O | 0.006 | 0.006 | 0.006 | 0.006 |
| KCl | 0.14 | 0.14 | 0.14 | 0.14 |
| NaCl | 0.25 | 0.25 | 0.25 | 0.25 |

TABLE 24

| | |
|---|---|
| Age of the product stored | |
| at 40° C. | Stabilized ClO₂, ppm |

| | | | | |
|---|---|---|---|---|
| 22 days | 59.9 | 57.6 | 59.8 | 57.7 |
| 49 days | 29.0 | 31.4 | 44.0 | 42.4 |
| 107 days | 8.5 | | | 11.6 |

A: In a solution that contains 0.8% Lumulse GRH-40, 1% flaxseed oil, 0.0072 stabilized ClO₂, 0.1% boric acid, 0.2% sorbitol, 0.69% 1N NaOH, 0.006% CaCl₂ 2H₂0, 0.006% MgCl₂ 6H₂0, 0.14% KCl, and 0.25% NaCl, stabilized ClO₂ levels drop to 59.9 ppm after 22 days, 29.0 ppm after 49 days and 8.5 ppm after 107 days.

B: In a solution that contains 1.0% Lumulse GRH-40, 1% flaxseed oil, 0.0072 stabilized ClO₂, 0.1% boric acid, 0.2% sorbitol, 0.69% 1N NaOH, 0.006% CaCl₂ 2H₂0, 0.006% MgCl₂ 6H₂0, 0.14% KCl, and 0.25% NaCl, stabilized ClO₂ levels drop to 57.6 ppm after 22 days and 31.4 ppm after 49 days.

C: In a solution that contains 1.2% Lumulse GRH-40, 1% flaxseed oil, 0.0072 stabilized ClO₂, 0.1% boric acid, 0.2% sorbitol, 0.69% 1N NaOH, 0.006% CaCl₂ 2H₂0, 0.006% MgCl₂ 6H₂0, 0.14% KCl, and 0.25% NaCl, stabilized ClO₂ levels drop to 59.8 ppm after 22 days and 44.0 ppm after 49 days.

D: In a solution that contains 1.5% Lumulse GRH-40, 1% flaxseed oil, 0.0072 stabilized ClO₂, 0.1% boric acid, 0.2% sorbitol, 0.69% 1N NaOH, 0.006% CaCl₂ 2H₂0, 0.006% MgCl₂ 6H₂0, 0.14% KCl, and 0.25% NaCl, stabilized ClO₂ levels drop to 57.7 ppm after 22 days, 42.4 ppm after 49 days and 11.6 ppm after 107 days.

### Example 15

### Oxidative Preservatives are not Compatible with Omega-3 Oil Emulsions

Table 25 also shows that initial levels of stabilized ClO₂ are reduced due to an interaction with Omega-3 fatty acids. Initial levels of 129 ppm ClO₂ are reduced after 86 days to 29 ppm.

TABLE 25

| FORMULATION | % w/w |
|---|---|
| Lumulse | 1.2 |
| Flax oil | 1 |
| Stablized ClO₂ | 0.0129 |
| Boric Acid | 0.6 |
| Sodium Borate | 0.035 |
| Taurine | 0.05 |
| CaCl₂.2H₂O | 0.006 |
| MgCl₂.6H₂O | 0.006 |
| KCl | 0.14 |
| NaCl | 0.35 |

TABLE 26

| Age of the product stored | Stabilized ClO₂, |
|---|---|
| at 40° C. | ppm |
| 22 days | 121.0 |
| 86 days | 29.0 |

In a solution that contains 1.2% Lumulse GRH-40, 1% flaxseed oil, 0.0129 stabilized ClO₂, 0.6% boric acid, 0.035% Sodium Borate, 0.05% Taurine, 0.2% sorbitol, 0.69% 1N NaOH, 0.006% CaCl₂.2H₂0, 0.006% MgCl₂.6H₂0, 0.14% KCl, and 0.25% NaCl, stabilized ClO₂ levels drop to 121.0 ppm after 22 days and 29.0 ppm after 86 days.

Thus, while oxidative preservatives, such as ClO₂, tend to be incompatible with stable Omega-3 emulsions, cationic antimicrobials, such as PHMB, unexpectedly both maintain emulsion stability and retain their preservative effects.

What is claimed is:

1. An ophthalmic composition comprising:
oil globules dispersed in an aqueous phase, said globules comprising:
(a) a surfactant component;
(b) a polar oil component comprising an Omega-3 fatty acid, wherein the surfactant to oil ratio is adjusted such that said oil globules have an average size of about 0.1 micron or less; and
(c) a viscosity modifying agent in a concentration that produces a viscosity at least as viscous as 0.25% 800K (w/w) Sodium Hyaluronate.

2. The composition of claim 1, wherein the surfactant component consists essentially of one or two surfactants.

3. The composition of claim 1, wherein the surfactant to oil ratio is adjusted to obtain oil globules having an average size of about 0.08 micron or less.

4. The composition of claim 1, wherein the surfactant to oil ratio is adjusted to obtain oil globules having an average size of about 0.05 micron or less.

5. The composition of claim 1, wherein the Omega-3 fatty acid component is selected from a natural oil that is a source of Omega-3 fatty acids.

6. The composition of claim 1, wherein the Omega-3 fatty acid component is selected from a synthetic oil that is a source of Omega-3 fatty acids.

7. The composition of claim 5, wherein the Omega-3 fatty acid component is from flaxseed oil.

8. The composition of claim 5, wherein the Omega-3 fatty acid component is from Perilla seed oil.

9. The composition of claim 1, wherein the composition is self-emulsifying.

10. The self-emulsifying composition of claim 9, wherein the surfactant component has a hydrophobic portion which comprises a first part oriented proximal to the aqueous phase that is larger than a second part of the hydrophobic portion of the surfactant component oriented towards the interior of the oil globule.

11. The self-emulsifying composition of claim 9, wherein the surfactant component consists essentially of one surfactant with the first part of the hydrophobic portion of the surfactant that contains more atoms than the second part of the hydrophobic portion of the surfactant.

12. The self-emulsifying composition of claim 9, wherein the surfactant component consists essentially of two surfactants, a first of said surfactants comprising a first hydrophobic portion and a second of said surfactants comprising a second hydrophobic portion, said first hydrophobic portion having a longer chain length than the second hydrophobic portion.

13. A self-emulsifying composition according to claim 9, further comprising an additional surfactant that does not interfere with self-emulsification.

14. The self-emulsifying composition of claim 9, wherein the surfactant component is selected from the group consisting of (a) a compound having at least one ether formed from at least about 1 to 100 ethylene oxide units and at least one fatty alcohol chain having from at least about 12 to 22 carbon atoms; (b) a compound having at least one ester formed from at least about 1 to 100 ethylene oxide units and at least one fatty acid chain having from at least about 12 to 22 carbon atoms; (c) a compound having at least one ether, ester or amide formed from at least about 1 to 100 ethylene oxide units and at least one vitamin or vitamin derivative; and (d) combinations thereof consisting of no more than two surfactants.

15. The self-emulsifying composition of claim 9, wherein the surfactant component is selected from the group consisting of Lumulse GRH-40 and TPGS.

16. The self-emulsifying composition of claim 9, wherein the surfactant component is Lumulse GRH-40.

17. The composition of claim 1, wherein the pH of the composition is in the range of about 6.5 to about 8.5.

18. The composition of claim 17, wherein the pH of the composition is in the range of about 7.3 to about 7.7.

19. The composition of claim 1, wherein the osmolality of the composition is from about 250 to about 330 mOsm/kg.

20. The composition of claim 19, wherein osmolality of the composition is from about 270 to about 310 mOsm/kg.

21. The composition of claim 1, formulated as a multipurpose solution for contact lenses.

22. A method of preparing the composition of claim 1 comprising:
preparing an oil phase comprising an Omega-3 fatty acid and a surfactant component, wherein the Omega-3 fatty acid and the surfactant component in the oil phase are in the liquid state;
preparing an aqueous phase at a temperature that permits self-emulsification;
wherein the aqueous phase comprises the viscosity modifying agent; and
mixing the oil phase and the aqueous phase to form an emulsion, without mechanical homogenization.

23. A method of preparing a composition according to claim 22, further comprising forming a milky paste or a clear viscous gel between the oil phase and a part of the aqueous phase and mixing the paste or gel with the rest of the aqueous phase to form an emulsion.

24. The method of preparing an ophthalmic composition according to claim 22, wherein the viscosity modifying agent is selected from the group consisting of hyaluronic acid and salts thereof, polyvinylpyrrolidone (PVP), cellulose polymers, including hydroxypropyl methyl cellulose (HPMC), hydroxyethyl cellulose (HEC), ethyl hydroxyethyl cellulose, hydroxypropyl cellulose, methyl cellulose and carboxymethyl cellulose (CMC), dextran 70, gelatin, glycerine, polyethylene glycols, polysorbate 80, propylene glycol and povidone.

25. The method of preparing an ophthalmic composition according to claim 22, wherein the viscosity modifying agent is selected from the group consisting of carbomers (e.g. carbopol RTM), polyvinyl alcohol, alginates, carrageenans, and guar, karaya, agarose, locust bean, tragacanth and xanthan gums.

26. The method of claim 24, wherein the cellulose polymer is carboxymethylcellulose (CMC) or hydroxypropyl methylcellulose.

27. The method of claim 24, wherein the polyethylene glycol is PEG 300 or PEG 400.

28. The method of preparing an ophthalmic composition according to claim 22, wherein the surfactant component consists essentially of one or two surfactants.

29. A method of treating dry eye in an individual comprising administering a composition according to claim 1 directly to an eye of the individual.

30. A method of treating dry eye in an individual comprising administering a composition according to claim 5 directly to an eye of the individual.

31. A method of treating dry eye in an individual comprising administering a composition according to claim 15 directly to an eye of the individual.

32. An ophthalmic composition comprising:
oil globules dispersed in an aqueous phase, said globules comprising:
(a) a surfactant component;
(b) a polar oil component comprising an Omega-3 fatty acid; and
(c) a polymeric quaternary amine preservative in an amount sufficient to produce a composition that meets U.S. preservative efficacy testing standards.

33. The composition of claim 32, wherein the polymeric quaternary amine preservative has an HLB value significantly higher than the HLB value of the polar oil component.

34. The composition of claim 32, wherein sufficient amounts of polymeric quaternary amine preservative are added to meet European preservative efficacy standards.

35. The composition of claim 32, wherein the polymeric quaternary amine preservative is selected from the group consisting of poly[dimethylimino-w-butene-1,4-diyl]chloride, alpha-[4-tris(2-hydroxyethyl)ammonium]dichloride (Polyquaternium 1®), poly(oxyethyl(dimethyliminio)ethylene dmethyliminio)ethylene dichloride (WSCP®), polyhexamethylene biguanide (PHMB), polyaminopropyl biguanide (PAPB).

36. The composition of claim 35, wherein the wherein the polymeric quaternary amine preservative is ispolyhexamethylene biguanide (PHMB).

37. The composition of claim 32, wherein the concentrations of the polymeric quaternary amine preservative range from about 0.00001% to about 2% (w/v).

38. The composition of claim 32, wherein the concentrations of the polymeric quaternary amine preservative range from about 0.00005% to about 1% (w/v).

39. The composition of claim 32, wherein the concentrations of the polymeric quaternary amine preservative is about 0.0001% (w/v).

40. The composition of claim 32, wherein the polymeric quaternary amine preservative is present at an ophthalmically acceptable or safe concentration such that the user can remove the disinfected lens from the composition and thereafter directly place the lens in the eye for safe and comfortable wear.

41. A method of treating dry eye in an individual comprising administering a composition according to claim 32 directly to an eye of the individual.

42. A method of treating dry eye in an individual comprising administering a composition according to claim 35 directly to an eye of the individual.

43. A method of treating dry eye in an individual comprising administering a composition according to claim 36 directly to an eye of the individual.
